SEQUENCE LISTING (14 sequences)

SEQ ID NO: 1 - protein, 5 aa, Artificial
  Chemically synthesized peptide derived from human growth hormone
LRIVQ

SEQ ID NO: 2 - protein, 6 aa, Artificial
  Chemically synthesized peptide derived from human growth hormone
YLRIVQ

SEQ ID NO: 3 - protein, 5 aa, Artificial
  Chemically synthesized peptide derived from human growth hormone
LRVIQ

SEQ ID NO: 4 - protein, 6 aa, Artificial
  Chemically synthesized peptide derived from human growth hormone
IVQXXX

SEQ ID NO: 5 - protein, 5 aa, Artificial
  Chemically synthesized peptide derived from human growth hormone
LKIVQ

SEQ ID NO: 6 - protein, 4 aa, Artificial
  Chemically synthesized peptide derived from human growth hormone
RIVQ

SEQ ID NO: 7 - protein, 5 aa, Artificial
  Chemically synthesized peptide derived from human growth hormone
LRIIQ

SEQ ID NO: 8 - protein, 5 aa, Artificial
  Chemically synthesized peptide derived from human growth hormone
LRVVQ

SEQ ID NO: 9 - protein, 6 aa, Artificial
  Chemically synthesized peptide derived from human growth hormone to be coupled to C-terminus of poorly available parent peptide
QVIRLY

SEQ ID NO: 10 - protein, 16 aa, Artificial
  Drug or parent peptide
GCCSDPRCNYDHPEIC

SEQ ID NO: 11 - protein, 34 aa, Artificial
  Drug or parent peptide
SVSEIQLMHNLGKHLNSMERVEWLRKKLQDVHNF

SEQ ID NO: 12 - protein, 22 aa, artificial
  Drug or parent peptide conjugated to chemically synthesized peptide derived from human growth hormone
YLRIVQGCCSDPRCNYDHPEIC

SEQ ID NO: 13 - protein, 22 aa, artificial
  Drug or parent peptide conjugated to synthetic peptide derived from human growth hormone
YLRIVQGCCSDPRCNYDHPEIC

SEQ ID NO: 14 - protein, 39 aa, artificial
  Drug or parent peptide conjugated to synthetic peptide derived from human growth hormone
LRIVQSVSEIQLMHNLGKHLNSMERVEWLRKKLQDVHNF

(19) United States
(12) Patent Application Publication
(10) Pub. No.: US 2011/0092448 A1
(43) Pub. Date: Apr. 21, 2011

(54) Peptide Conjugate for Oral Delivery of Hydrophilic Peptide Analgesics

(75) Inventor: Christopher Ian Belyea, Elsternwick (AU)

(73) Assignee: METABOLIC PHARMACEUTICALS LIMITED, Melbourne (AU)

(21) Appl. No.: 11/989,906

(22) PCT Filed: Aug. 2, 2006
(86) PCT No.: PCT/AU2006/001098

§ 371 Date: Jul. 2, 2008

(30) Foreign Application Priority Data
Aug. 2, 2005 (AU) 2005904150

(51) Int. Cl. A61K 38/16; C07K 5/08; C07K 14/00; C07K 7/06; C07K 7/08; C07K 5/10; A61K 38/08; A61K 38/10; A61P 3/10; A61P 5/06; A61P 25/00

(52) U.S. Cl. 514/21.2; 530/331; 530/326; 530/324; 530/329; 530/328; 530/327; 530/325; 530/350; 530/330; 514/21.8; 514/21.7; 514/21.6; 514/21.5; 514/21.4; 514/21.3

(57) ABSTRACT

The present invention provides a method of improving the oral delivery of a parent peptide, comprising the step of linking the parent peptide to an added peptide to form a conjugate which has greater oral bioavailability than the parent molecule alone, the added peptide comprising a balance of hydrophobic and hydrophilic residues as defined herein. Conjugates for use in the method are also provided, as are pharmaceutical compositions comprising the conjugate and methods of treatment using the conjugate or pharmaceutical composition.

The invention relates to methods for improving the oral delivery of proteins or peptides, and in particular to methods of designing proteins or peptides with improved bioavailability when administered orally and proteins and peptides which have improved oral bioavailability.

### BACKGROUND OF THE INVENTION

Oral administration of therapeutic agents is desirable because it is generally associated with optimal compliance by the patient with the treatment regimen, and permits greater flexibility of the dosing schedule, as well as avoiding the risks, inconvenience and expense associated with administration by injection. However, the ability to utilize the oral route is limited by the ability of the drug:

(a) to be absor [symbol] es that the balance of in the oral cavity, oesoph [symbol] residues provided by the added

(b) and to survive acid and enzymic degradation in the digestive tract, and

(c) to pass across the epithelial cell layer into the systemic circulation.

Almost all pharmacological peptides are not orally available to a useful extent. In particular, hormones such as insulin, growth hormone, follicle-stimulating hormone or calcitonin, and cytokines such as interferon or interleukin, are known to have oral availabilities without special formulation well below 2%. At such levels, the temporal and inter-individual variability in availability is typically high, rendering oral administration impractical, uneconomical or dangerous.

Peptides are of increasing importance in medical treatment. However, their use has been limited by the fact that the great majority of peptides have to be administered by injection. Although alternative routes of systemic administration have been suggested, such as the pulmonary, nasal or transdermal routes, hitherto these have been developed only for a limited range of agents and suffer from limitations in tolerability and in the amount of compound that can be delivered in a single dose.

Various attempts have been made to improve the bioavailability of pharmaceuticals. These include incorporation of penetration enhancers, such as salicylates, lipid-bile salt mixes, micelles, glycerides and acylcarnitines but these are found to cause toxicity problems on most occasions.

If the pharmaceutical is a protein or peptide, attempts to improve oral bioavailability include mixing the protein or peptide with protease inhibitors such as aprotinin, soybean trypsin inhibitor and amastatin, to limit degradation of the administered therapeutic agent. Unfortunately these protease inhibitors are not selective and endogenous proteases are also inhibited by them with undesirable effects.

Other attempts to provide oral formulations of peptides have utilized protective coatings such as enteric coatings, alone or together with chemical modification of the peptide by coupling of the protein or peptide to amphiphilic oligomers or polymers comprising for example a hydrophilic polyethylene glycol moiety and a lipophilic alkyl moiety. These techniques confer very limited success. Another approach is to add an excipient that loosens the tight junctions in the gastrointestinal tract, but this approach causes tolerability problems, because the compromised barrier may admit all molecules in the vicinity, including bacteria. Also calcium alginate-coated liposome formulations have been used for colonic delivery of peptides. However, so far such approaches have found only limited application.

There is therefore a need in the art for new methods for improving the oral delivery of molecules, especially proteins or peptides.

### SUMMARY OF THE INVENTION

In a first aspect, the invention provides a method of improving the oral delivery of a parent peptide, comprising the step of linking the parent peptide to an added peptide to form a conjugate which has greater oral bioavailability than the parent molecule alone, the added peptide comprising a peptide of formula I

A-B-C (I)

in which A and C are each a hydrophobic amino acid residue or a substantially hydrophobic peptide of between 2 and 9 amino acid residues, A and C may be different and one of A or C may be absent
and B is one or more hydrophilic amino acid residues.

The applicant's earlier applications PCT/AU98/00724, PCT/AU01/00354 and PCT/AU00/01362, describe a family of peptides derived from the C-terminal sequence of human growth hormone, especially amino acid residues 177-199, and analogues of this sequence. The peptides in this family, termed AOD peptides, have been found to have no known toxicity at any dose, and can be effectively administered at frequencies ranging from once every few days to continuously. Some of the AOD peptides, including AOD9604 and AOD9401 as described in these applications, have been found to be substantially orally bioavailable. The inventor has now found that linking a subregion of the AOD peptides to a peptide which is not itself orally bioavailable (or only has limited oral bioavailability) can confer substantial oral bioavailability upon that peptide. Further analysis of the subregion of the AOD peptides has allowed the inventor to determine the essential components of an added peptide required to improve the oral delivery of a parent peptide to which the added peptide is linked.

In a second aspect, the invention provides an oral delivery system comprising an added peptide, the added peptide comprising a peptide of formula I

A-B-C (I)

in which A and C are each a hydrophobic amino acid residue or a substantially hydrophobic peptide of between 2 and 9 amino acid residues, A and C may be different and one of A or C may be absent and B is one or more hydrophilic amino acid residues, wherein the added peptide is for conjugating to a parent peptide to improve the oral bioavailability of the parent peptide.

In a third aspect the invention provides a peptide conjugate comprising a parent peptide linked to an added peptide, the added peptide comprising a peptide of formula I

A-B-C (I)

in which A and C are each a hydrophobic amino acid residue or a substantially hydrophobic peptide of between 2 and 9 amino acid residues, A and C may be different and one of A or C may be absent
and B is one or more hydrophilic amino acid residues.

In a fourth aspect, the invention provides a pharmaceutical composition for oral administration, comprising a conjugate according to the third aspect of the invention, together with a pharmaceutically-acceptable carrier.

In a fifth aspect the present invention provides for the use of a peptide comprising a peptide of formula I

A-B-C (I)

in which A and C are each a hydrophobic amino acid residue or a substantially hydrophobic peptide of between 2 and 9 amino acid residues, A and C may be different and one of A or C may be absent
and B is one or more hydrophilic amino acid residues, for linking to a parent peptide to improve the oral bioavailability of the parent peptide.

In a sixth aspect the present invention also provides methods of preventing or treating a pathological disorder in an animal in need of treatment with the parent peptide, by orally administering to the animal an effective amount of a conjugate according to the third aspect of the invention or a pharmaceutical composition according to the fourth aspect of the invention, in which the parent peptide is not normally substantially orally bioavailable.

The seventh aspect of the present invention provides use of an added peptide comprising a peptide of formula I

A-B-C (I)

in which A and C are each a hydrophobic amino acid residue or a substantially hydrophobic peptide of between 2 and 9 amino acid residues, A and C may be different and one of A or C may be absent
and B is one or more hydrophilic amino acid residues, in the manufacture of a medicament comprising a parent peptide, in which the medicament is for administering orally to a patient in need of treatment with the parent peptide.

It is also contemplated that the added peptide may be used to orally deliver parent peptides for use in diagnosis and or monitoring the progression of a pathological disorder and or the effect of a further therapeutic agent on the progression of the pathological disorder.

Without wishing to be limited by any proposed mechanism for the observed beneficial effect, it is thought that the added peptide has particular membrane binding properties and lipophilic and hydrophilic balance which enhance transport of the conjugate across the mucosal layers in the gastrointestinal (GI) tract to enable encounter with, binding to and transport across the epithelial cell membrane.

### BRIEF DESCRIPTION OF THE FIGURES

FIG. 1 shows the results of assays of the effects of ACV1 and ACV3 on stimulation of catecholamine release from bovine chromaffin cells in vitro.

(a) ACV1 (mean of two determinations)

(b) ACV3 (mean of two determinations).

FIG. 2 shows a comparison of the effect of ACV1 in a model of neuropathic pain in rats.

(a) Subcutaneous injection, 20 μg/kg body weight

(b) Sublingual administration, 100 μg/kg body weight

(c) Oral administration, 100 μg/kg body weight.

FIG. 3 shows the effect of sublingual administration of ACV3 in a model of neuropathic pain in rats.

FIG. 4 shows the comparative effect of oral and subcutaneous administration of ACV3 in STZ rats, another model of neuropathic pain.

(a) Subcutaneous injection of ACV3

(b) Oral administration of ACV3

FIG. 5 shows the effect of orally administered ACV3.2 in STZ rats.

FIG. 6 shows the effects of orally administered EP-PTH on blood calcium levels in mice.

### DETAILED DESCRIPTION OF THE INVENTION

The inventor proposes that the balance of hydrophobic and hydrophilic residues provided by the added peptide allows the added peptide to protect the parent peptide from acid and enzymic degradation in the digestive tract and to allow the parent peptide to be absorbed through the mucosal layers in the oral cavity, oesophagus or gut, and to pass across the epithelial cell layer into the systemic circulation, without removing the therapeutic activity of the parent peptide.

Use of the term “peptide” in this specification includes polypeptides of any amino acid length, including proteins, unless specifically restricted.

Accordingly, as defined herein the parent peptide encompasses parent peptides, polypeptides and proteins.

As referred to herein “oral delivery” or “oral administration” are intended to encompass any administration or delivery to the GI tract and includes administration directly to the oropharyngeal cavity, and administration via the mouth in which the actual absorption of the peptide or polypeptide takes place in the gastrointestinal tract, including the stomach, small intestine, or large intestine. Oral administration as used herein encompasses sublingual administration (administration by application under the tongue of the recipient, representing one form of administration via the oropharyngeal cavity) and buccal administration (administration of a dosage form between the teeth and the cheek of the recipient).

Oral delivery and oral administration may be used interchangeably herein.

Bioavailability as used herein refers to the availability of the parent peptide in the bloodstream.

### Added Peptide

In the added peptide of formula I, A and C may be the same or different and one of either A or C may be absent. A and or C are either a hydrophobic amino acid residue or a substantially hydrophobic peptide of 2-9 amino acid residues.

A hydrophobic amino acid as defined herein is a naturally occurring amino acid residue selected from Alanine, Valine, Leucine, Isoleucine, Proline, Phenylalanine, Tyrosine, Cysteine, Tryptophan and Methionine or is an analogue thereof as defined below

A substantially hydrophobic peptide as defined herein is a peptide having a sequence which includes at least 1 hydrophobic amino acid and which does not include any hydrophilic or charged amino acids. Preferably, at least 50% of the amino acids make up the hydrophobic peptide sequence are hydrophobic amino acids. More preferably at least 80% of all amino acids which make up the hydrophobic peptide sequence are hydrophobic amino acids. More preferably at least 90% or 95% of all amino acids which make up the hydrophobic peptide sequence are hydrophobic amino acids. Preferably the remainder of the hydrophobic residue comprises neutral amino acids.

A hydrophilic amino acid residue as defined herein is a naturally occurring amino acid residue selected from Serine or Threonine or a naturally occurring charged amino acid residue, or is an analogue thereof as defined below.

A charged amino acid as defined herein is a naturally occurring amino acid residue selected from Arginine, Lysine and Histidine (all positively charged or basic) and Aspartic acid and Glutamic acid (both negatively charged or acidic) or is an analogue thereof as defined below.

A neutral amino acid as defined herein is a naturally occurring amino acid residue selected from Glycine, Asparagine or Glutamine or is an analogue thereof as defined below.

A naturally occurring amino acid residue is a L-amino acid residue. These are generally termed “common amino acids” and are selected from the group consisting of Glycine, Leucine, Isoleucine, Valine, Alanine, Phenylalanine, Tyrosine, Tryptophan, Aspartic acid, Asparagine, Glutamic acid, Glutamine, Cysteine, Methionine, Arginine, Lysine, Proline, Serine, Threonine and Histidine. These are referred to herein by their conventional three-letter or one-letter abbreviations.

It is contemplated that one or more of the individual amino acid residues within the sequence of the added peptide may be substituted by a non-conventional amino acid (i.e. an analogue or uncommon amino acid) provided that the conformation, structure and charge of the added peptide is sufficiently retained to permit the added peptide to confer an improvement in oral delivery to the parent peptide to which it is linked.

An analogue of a naturally occurring amino acid as used herein includes non-conventional amino acids or chemical amino acid analogues. Thus for example Leucine may be replaced by Norleucine, Valine may be replaced by Norvaline, Cysteine may be replaced by Homocysteine, Serine may be replaced by Homoserine, Lysine may be replaced by 5-Hydroxylysine, Proline by 4-Hydroxyproline, Arginine may be replaced by Homoarginine, Ornithine or Citrulline, Alanine may be replaced by α-Methylalanine or β-Alanine, a D-amino acid may be used instead of the corresponding L-amino acid, any amino acid may be N-methylated, or the N-terminus may be acetylated. A non-conventional amino acid further includes one selected from the group consisting of D-amino acids, homo-amino acids, N-alkyl amino acids, dehydroamino acids, aromatic amino acids (other than phenylalanine, tyrosine and tryptophan), ortho-, meta- or para-aminobenzoic acid, ornithine, citrulline, norleucine, γ-glutamic acid, aminobutyric acid (Abu), and α,α-disubstituted amino acids.

Non-conventional amino acids also include compounds which have an amine and carboxyl functional group separated in a 1,3 or larger substitution pattern, such as β-alanine, γ-amino butyric acid, Freidinger lactam, the bicyclic dipeptide (BTD), amino-methyl benzoic acid and others well known in the art. Statine-like isosteres, hydroxyethylene isosteres, reduced amide bond isosteres, thioamide isosteres, urea isosteres, carbamate isosteres, thioether isosteres, vinyl isosteres and other amide bond isosteres known to the art may also be used.

The use of analogues or non-conventional amino acids may improve the stability and biological half-life of the added peptide since they are more resistant to breakdown under physiological conditions. The person skilled in the art will be aware of similar types of substitution which may be made.

A non limiting list of non-conventional amino acids which may be used as suitable replacements for the naturally occurring amino acids and their standard abbreviations is set out in Table 1.

TABLE 1
Non-conventional amino acids

| Non-conventional | | Non-conventional | |
|---|---|---|---|
| amino acid | Abbrev. | amino acid | Abbrev. |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | arg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl- | Nmbc | L-O-methyl serine | Omser |
| ethylamino)cyclopropane | | L-O-methyl homoserine | Omhser |

It is to be clearly understood that the invention also encompasses analogues of the added peptide which include but are not limited to the following:

1. peptides in which one or more amino acids is replaced by its corresponding D-amino acid. The skilled person will be aware that such sequences, including retro-inverso amino acid sequences where substantially all of the amino acids are D-amino acids and the order is reversed can be synthesised by standard methods; see for example Chorev and Goodman, (1993) Acc. Chem. Res., 26, 266-273,

2. Peptidomimetic compounds, in which the peptide bond is replaced by a structure more resistant to metabolic degradation. See for example Olson et al., (1993) J. Med. Chem. 36 3039-3049; and

3. peptides in which individual amino acids are replaced by analogous structures, for example, gem-diaminoalkyl groups or alkylmalonyl groups, with or without modified termini or alkyl, acyl or amine substitutions to modify their charge.

In a preferred embodiment of the present invention A and or C is a hydrophobic amino acid residue. Alternatively A and or C is a substantially hydrophobic peptide comprising 2, 3, 4, 5, 6, 7, 8 or 9 amino acids.

Preferably B is a maximum of 5 hydrophilic amino acids, more preferably 4, 3, or 2 hydrophilic amino acids. Most preferably B is a hydrophilic residue. In a preferred embodiment B is between 1 to 5 charged amino acid residues, and is more preferably 1 to 5 basic amino acid residues. Preferably B is L- or D-Arg, His or Lys.

In one embodiment A or C have the sequence

Ile-Val-Gln-Xₐ-X_{b}-X_{c}, in which each of Xₐ-X_{c} is any non-hydrophilic amino acid.

The added peptide preferably has a maximum length of 12 amino acid residues, more preferably 10 amino acid residues and most preferably 5, 6, 7, 8, or 9 amino acid residues.

Preferred added peptides comprise one of the sequences:

| | |
|---|---|
| Leu-Arg-Ile-Val-Gln- | (SEQ ID NO: 1) |
| Tyr-Leu-Arg-Ile-Val-Gln-. | (SEQ ID NO: 2) |
| Leu-Arg-Val-Ile-Gln- | (SEQ ID NO: 3) |
| Leu-Arg-Ile-Val-Gln, | (SEQ ID NO: 4) |
| Leu-Lys-Ile-Val Gln-, | (SEQ ID NO: 5) |
| Arg-Ile-Val-Gln-, | (SEQ ID NO: 6) |
| Leu-Arg-Ile-Ile-Gln-, | (SEQ ID NO: 7) |
| Leu-Arg-Val-Val-Gln-, | (SEQ ID NO: 8) |

or an analogue thereof. Preferably all amino acids, except for glycine, are of the L-absolute configuration.

### Parent Peptide

A parent peptide as used herein is a therapeutic peptide (or polypeptide or protein) which has no or limited oral bioavailability.

Preferably the parent peptide is sufficiently stable in the GI tract but has difficulty in entering the bloodstream.

Sufficiently stable as used herein refers at least 20% of the administered peptide remaining after 30 minutes of exposure in the GI tract. An example of such a peptide is the conotoxin ACV1, which is described in more detail below.

Parent peptides include enzymes, hormones, antibodies and antibody fragments and conotoxins.

The parent peptide may be a disulfide-bonded peptide such as a conotoxin, such as the one described in the examples. Such disulphide bonded peptides may have a particular advantage in that the disulphide bonds typically protect the parent peptide somewhat from degradation by gastrointestinal enzymes.

It is to be expected that as the size of the parent peptide increases, the oral availability conferred by the invention decreases.

The parent peptide may be a peptide of 20 or fewer amino acids, potentially providing substantial oral activity when administered using the oral delivery system of the present invention. Examples of such parent peptides include α-melanocyte stimulating hormone, vasopressin, oxytocin, enkephalin, somatostatin and conotoxins including ACV1.

The parent peptide may be between 21 and 40 amino acids, for example parathyroid hormone (PTH 1-34) as described in the examples for which the oral availability conferred by the delivery system of the invention may be less but still serviceable. Other examples of such parent peptides include glucagon-like peptide (GLP-1), calcitonin, PYY3-36, oxyntomodulin, Gastric Inhibitory Peptide (GIP), endorphin, and related members of the superfamily.

The parent peptide may be between 41 and 60 amino acids, for which the oral availability conferred by delivery system of the invention may be even less. Examples of such parent peptides are insulin and Insulin Like Growth Factor-1 (IGF-I).

The parent peptide may be between 61 and 80 amino acids, for which the oral availability conferred by delivery system of the invention may be even less.

The parent peptide may be greater than 80 amino acids, for which the oral availability conferred by delivery system of the invention may be expected to be relatively low at less than 2% and useful in only a few examples. Possible examples of such parent peptides include growth hormone, an interleukin, or other large growth factor.

Any amino acid of the parent peptide may be substituted by an analogue or non-conventional amino acid as described above in relation to the added peptide.

### Linkage

To improve the oral bioavailability of the parent peptide the added peptide must be linked to the parent peptide. The linkage may be a covalent or non-covalent linkage.

As is shown in the examples linking the added peptide to the parent peptide in accordance with the first aspect of the invention improves the oral bioavailability of the parent peptide. The added peptide can be used in a delivery system for oral delivery of any parent peptide as herein defined.

Preferably, the added peptide is attached at the N terminus of the parent peptide. It is further contemplated that the added peptide can be linked to the parent peptide or nucleotide at its C-terminus. In this embodiment the order of the amino acids in the added peptide is preferably reversed, and the C-terminus is preferably amidated. For example, in the embodiment above, the added peptide is:

| | |
|---|---|
| Tyr-Leu-Arg-Ile-Val-Gln | (SEQ ID NO: 2) |

If this peptide was added to the C-terminus of the parent peptide is preferably:

| | |
|---|---|
| Gln-Val-Ile-Arg-Leu-Tyr-NH2 | (SEQ ID NO: 9) |

The parent peptide and the added peptide may be linked by any convenient method which confers bioactivity. For example, if both the parent peptide and the added peptide are short enough to be conveniently synthesised by conventional solid-phase methods, such as those using Fmoc or Boc chemistry, this provides a very convenient method of synthesis and the linkage can be a conventional peptide bond. Alternatively, the parent peptide may be synthesised using recombinant methods, and subsequently isolated and linked to the added peptide using enzymic methods, or the whole conjugate may be synthesized using recombinant methods. Suitable methods will be well known to those skilled in the art, and the most convenient method for any given situation can be readily selected.

The preferred linkage site may vary, depending on the nature of the parent peptide. In one particular embodiment the conjugate in which the N-terminus of the parent peptide is linked to the C-terminus of the added peptide, but this will depend in the main on which addition point preserves bioactivity.

In one embodiment the added peptide comprises the sequence

Tyr-Leu-Arg-Ile-Val-Gln (SEQ ID NO: 2), and the parent peptide comprises the ACV1 sequence Gly-Cys-Cys-Ser-Asp-Pro-Arg-Cys-Asn-Tyr-Asp-His-Pro-Glu-Ile-Cys-NH₂ (SEQ ID NO: 10). The N terminus may optionally be acetylated.

In another embodiment the added peptide also comprises the sequence Leu-Arg-Ile-Val-Gln (SEQ ID NO: 3), and the parent peptide comprises the sequence PTH 1-34 sequence Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Lys-Leu-Gln-Asp-Val-His-Asn-Phe (SEQ ID NO: 11)

### Conjugate Peptide

A conjugate peptide as defined herein comprises an added peptide as defined herein linked to a parent peptide as defined herein, in any order.

Preferred conjugate peptides are those comprising or consisting essentially of SEQ ID Nos: 12, 13 or 14.

It will be clearly understood that the invention encompasses conjugate peptides in which the sequence is modified by one or more amino acid substitutions, deletions or additions, as described below.

Substitutions encompass amino acid alterations in which an amino acid is replaced with a different naturally-occurring or a non-conventional amino acid residue. Such substitutions may be classified as “conservative”, in which case an amino acid residue present in a peptide is replaced with another naturally-occurring amino acid of similar character, for example Gly to Ala, Asp to Glu, Asn to Gln or Trp to Tyr. Possible alternative amino acids include Serine or Threonine, Aspartic acid or Glutamic acid or γ-Carboxyglutamate, Proline or Hydroxyproline, Arginine or Lysine, Asparagine or Histidine, Histidine or Asparagine, Tyrosine or Phenylalanine or Tryptophan, Aspartate or Glutamate, Isoleucine or Leucine or Valine.

Such conservative substitutions are shown in Table 2 under the heading of preferred substitutions. If such substitutions do not result in a change in functional activity, then more substantial changes, denoted exemplary substitutions in Table 2, or as further described below in reference to amino acid classes, may be introduced, and the resulting variant analyzed for functional activity.

TABLE 2
Amino acid substitutions

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro | pro |
| His (H) | asn; gln; lys; arg | arg |
| Ile (I) | leu; val; met; ala; phe; | leu |
| | norleucine | |
| Leu (L) | norleucine; ile; val; | ile |
| | met; ala; phe | |
| Lys (K) | arg; gln; asn | arg |
| Met (M) | leu; phe; ile | leu |
| Phe (F) | leu; val; ile; ala | leu |
| Pro (P) | gly | gly |
| Ser (S) | thr | thr |
| Thr (T) | ser | ser |
| Trp (W) | tyr | tyr |
| Tyr (Y) | trp; phe; thr; ser | phe |
| Val (V) | ile; leu; met; phe; | leu |
| | ala; norleucine | |

Substitutions encompassed by the present invention may also be “non-conservative”, in which an amino acid residue which is present in a polypeptide is substituted with an amino acid having different properties, such as a naturally-occurring amino acid from a different group (e.g. substituting a charged or hydrophilic or hydrophobic amino acid with Alanine), or alternatively, in which a naturally-occurring amino acid is substituted with a non-conventional amino acid. Additions encompass the addition of one or more naturally occurring or non-conventional amino acid residues. Deletions encompass the deletion of one or more amino acid residues.

Without wishing to limit the scope of the invention, it is presently believed that any residues which are involved in disulphide bonds in the parent peptide, and in particular the cysteine residues of conotoxins such as ACV1, are likely to be essential to the biological activity of the molecule, and therefore the scope of substitution at these points may be limited.

Methods for combinatorial synthesis of peptide analogues and for screening of peptides and peptide analogues to determine that they retain activity are well known in the art (see for example Gallop et al., (1994) J. Med. Chem. 37, 1233-1251; Hogan (1997) Nature Biotechnology, 15 328-330.

The conjugate can be modified either during or after synthesis, by methods including but not limited to glycosylation, acetylation, phosphorylation, amidation, etc.

The term “AOD peptide” as used herein refers to a member of the class of peptides derived from the C-terminal sequence of human growth hormone, and especially from amino acid residues 177-199 or human growth hormone, as discussed above.

The term “ACV1 peptide” means the α-conotoxin disclosed in International Patent Application No. PCT/AU02/00411 as Vc1.1, having the sequence provided as SEQ ID NO:10.

### Pharmaceutical Compositions

An aspect of the invention provides various pharmaceutical compositions useful for preventing or treating pathological conditions. The pharmaceutical compositions according to one embodiment of the invention are prepared by bringing a peptide conjugate according to the third aspect of the invention, or an analogue, derivative or salt thereof, into a form suitable for administration to a subject, using carriers, excipients and additives or auxiliaries.

Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Other pharmaceutically acceptable carriers include non-toxic excipients, including salts, preservatives, buffers and the like, as described in Remington's Pharmaceutical Sciences, 20th ed. Williams & Wilkins (2000) and The British National Formulary 43rd ed. (British Medical Association and Royal Pharmaceutical Society of Great Britain, 2002; http://bnf.rhn.net), the contents of which are hereby incorporated by reference.

Preservatives include antimicrobials, anti-oxidants, and chelating agents. The pH and exact concentration of the various components of the pharmaceutical composition are adjusted according to routine skills in the art. See Goodman and Gilman's The Pharmacological Basis for Therapeutics (7th ed., 1985).

The pharmaceutical compositions are preferably prepared and administered in dosage units. Solid dosage units include tablets, capsules and suppositories. For treatment of a subject, depending on activity of the compound, manner of administration, nature and severity of the disorder, age and body weight of the subject, different daily doses can be used. Under certain circumstances, however, higher or lower daily doses may be appropriate. The administration of the daily dose can be carried out both by single administration in the form of an individual dose unit or else several smaller dose units and also by multiple administration of subdivided doses at specific intervals.

The pharmaceutical compositions of the invention may benefit from an enteric coating to reduce degradation in the GI tract. Since the preferred parent peptide is substantially stable in the GI tract an enteric coating will not be necessary in all circumstances.

The pharmaceutical compositions according to the invention may be administered in a therapeutically effective dose. Amounts effective for this use will, of course, depend on the severity of the disease and the weight and general state of the subject. Typically, dosages used in vitro may provide useful guidance in the amounts useful for in situ administration of the pharmaceutical composition, and animal models may be used to determine effective dosages for treatment of the cytotoxic side effects. Various considerations are described, e.g. in Langer, Science, 249: 1527, (1990).

Formulations for oral use may be in the form of hard gelatin capsules, in which the active ingredient is mixed with an inert solid diluent, for example, calcium carbonate, calcium phosphate or kaolin. They may also be in the form of soft gelatin capsules, in which the active ingredient is mixed with water or an oil medium, such as peanut oil, liquid paraffin or olive oil.

Aqueous suspensions are also suitable for oral use, and normally contain the active materials in admixture with excipients suitable for the manufacture of aqueous suspensions, for example saline. Such excipients may be suspending agents such as sodium carboxymethyl cellulose, methyl cellulose, hydroxypropylmethylcellulose, sodium alginate, polyvinylpyrrolidone, gum tragacanth and gum acacia; dispersing or wetting agents, which may be

(a) a naturally occurring phosphatide such as lecithin;

(b) a condensation product of an alkylene oxide with a fatty acid, for example, polyoxyethylene stearate;

(c) a condensation product of ethylene oxide with a long chain aliphatic alcohol, for example, heptadecaethylenoxycetanol;

(d) a condensation product of ethylene oxide with a partial ester derived from a fatty acid and hexitol such as polyoxyethylene sorbitol monooleate, or

(e) a condensation product of ethylene oxide with a partial ester derived from fatty acids and hexitol anhydrides, for example polyoxyethylene sorbitan monooleate.

Dosage levels of the conjugate of the present invention will vary widely depending on the potency of the conjugate, usually be of the order of about 1 μg to about 5 mg per kilogram body weight, from about 100 μg to about 500 mg per patient per day). The amount of active ingredient which may be combined with the carrier materials to produce a single dosage will vary, depending upon the host to be treated and the particular mode of administration. For example, a formulation intended for oral administration to humans may contain about 100 μg to 500 mg of an active compound with an appropriate and convenient amount of carrier material, which may vary from about 5 to 95 percent of the total composition. Dosage unit forms will generally contain between from about 5 mg to 500 mg of active ingredient.

It will be understood, however, that the specific dose level for any particular patient will depend upon a variety of factors including the activity of the specific compound employed, the age, body weight, general health, sex, diet, time of administration, route of administration, rate of excretion, drug combination and the severity of the particular disease undergoing therapy.

The compounds of the invention may additionally be combined with other compounds to provide an operative combination. It is intended to include any chemically compatible combination of pharmaceutically-active agents, as long as the combination does not eliminate the activity of the conjugate of this invention.

### Methods of Treatment

The conjugate or pharmaceutical compositions of the present invention may be used in methods of treatment of any pathological disorder which may be treated by the parent peptide, in which the parent peptide is administered orally.

Reference herein to treatment is intended to encompass prevention of the pathological disorder or alleviation of the pathological disorder.

The pathological disorder to be treated by the present invention may be any disorder which is treated by peptides. For example peptide drugs are known to treat diabetes (e.g. insulin), growth hormone deficiency (e.g. growth hormone), pain (e.g. conotoxins), etc.

In the description of the invention and in the claims which follow, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

As used herein, the singular forms “a”, “an”, and “the” include the corresponding plural reference unless the context clearly dictates otherwise. Thus, for example, a reference to “a peptide” includes a plurality of such peptides, and a reference to “an amino acid” is a reference to one or more amino acids.

Where a range of values is expressed, it will be clearly understood that this range encompasses the upper and lower limits of the range, and all values in between these limits.

Unless defined otherwise, all technical and scientific terms used herein have the same meaning as commonly understood by one of ordinary skill in the art to which this invention belongs. Although any materials and methods similar or equivalent to those described herein can be used to practice or test the present invention, the preferred materials and methods are described.

It is to be clearly understood that this invention is not limited to the particular materials and methods described herein, as these may vary. It is also to be understood that the terminology used herein is for the purpose of describing particular embodiments only, and it is not intended to limit the scope of the present invention, which will be limited only by the appended claims.

Unless otherwise indicated, the present invention employs conventional chemistry, protein chemistry, molecular biological and enzymological techniques within the capacity of those skilled in the art. Such techniques are well known to the skilled worker, and are explained fully in the literature. See, for example, Coligan, Dunn, Ploegh, Speicher and Wingfield: “Current protocols in Protein Science” (1999) Volumes I and II (John Wiley & Sons Inc.); Sambrook, Fritsch and Maniatis: “Molecular Cloning: A Laboratory Manual” (2001); Shuler, M. L.: Bioprocess Engineering: Basic Concepts (2nd Edition, Prentice-Hall International, 1991); Glazer, A. N., DeLange, R. J., and Sigman, D. S.: Chemical Modification of Proteins (North Holland Publishing Company, Amsterdam, 1975); Graves, D. J., Martin, B. L., and Wang, J. H.: Co- and post-translational modification of proteins: chemical principles and biological effects (1994); Lundblad, R. L. (1995) Techniques in protein modification. CRC Press, Inc. Boca Raton, Fl. USA; and Goding, J. W Monoclonal Antibodies: principles and practice (Academic Press, New York: 3rd ed. 1996).

The invention will now be described in detail by way of reference only to the following non-limiting examples and drawings.

### Example 1

### Synthesis of Peptides

Peptides were synthesised under contract to Metabolic Pharmaceuticals Limited by Auspep Pty Limited (Parkville, Australia) or Global Peptide (Colorado, USA), using conventional solid phase peptide synthetic methods and Fmoc chemistry (Barany and Albericcio (1991) Peptide synthesis for biotechnology in the 19990s In Bond, S (ed) Biotechnology International 1990/1991. London, Century press, pages 155-163).

The molecular weight of each peptide product was confirmed by mass spectrometric analysis, and the purity of the peptides was assessed by high-performance liquid chromatography, for example at Auspep Pty Ltd on a Merck Superspher® ²⁵⁰⁻⁴ LiChroCART 100 RP-18 column, using the following solvents: Solvent A—0.1% trifluorocetic acid in water; Solvent B—90% acetonitrile in water containing 0.1% trifluoroacetic acid. Elution was with a linear gradient of 100% A: 0% B to 30% A: 70% B over 35 min, at a flow rate of 1.0 ml/min. The eluate was monitored at 218 nm, and a homogenous single peak was obtained. All peptides were supplied in the form of the acetate salt. The purity of the products were at least 95%.

The following peptides were synthesised:

ACV1:
(SEQ ID NO: 10)
Gly-Cys-Cys-Ser-Asp-Pro-Arg-Cys-Asn-Tyr-Asp-His-
Pro-Glu-Ile-Cys-NH₂
ACV3:
(SEQ ID NO: 12)
Ac-Tyr-Leu-Arg-Ile-Val-Gly-Cys-Cys-Ser-Asp-Pro-
Arg-Cys-Asn-Tyr-Asp-His-Pro-Glu-Ile-Cys-NH₂
ACV3.2:
(SEQ ID NO: 13)
Tyr-Leu-Arg-Ile-Val-Gly-Cys-Cys-Ser-Asp-Pro-Arg-
Cys-Asn-Tyr-Asp-His-Pro-Glu-Ile-Cys-NH₂
EP-PTH:
(SEQ ID NO: 14)
Leu-Arg-Ile-Val-Gln-Ser-Val-Ser-Glu-Ile-Gln-Leu-
Met-His-Asn-Leu-Gly-Lys-His-Leu-Asn-Ser-Met-Glu-
Arg-Val-Glu-Trp-Leu-Arg-Lys-Lys-Leu-Gln-Asp-Val-
His-Asn-Phe-OH

ACV3 is an analogue of ACV1, consisting of an acetylated fragment of the N-terminus of AOD9604 attached by its C terminus to the N-terminus of ACV1. The Cysteine residues in all ACV peptides are in the conformation 1-3, 2-4, which forms spontaneously by air oxidation. ACV3.2 is the same as ACV3 other than being non-acetylated.

EP-PTH is an analogue of PTH 1-34, with the added peptide of SEQ ID NO:1 at the N-terminus of PTH 1-34.

### Example 2

### Effect of ACV Peptides on Nicotine-Evoked Release of Catecholamines from Chromaffin Cells

International Patent Application No. PCT/AU02/00411 describes a family of α-conotoxins, collectively referred to herein as “ACV peptides”, with unexpectedly powerful analgesic activity and, in at least one case, designated Vc1.1 in PCT/AU02/00411, the ability to accelerate recovery from nerve injury. A post-translational modification of this peptide lacks analgesic activity, but retains the ability of the parent compound to accelerate recovery from nerve injury.

Hitherto it has been necessary to administer conotoxins by injection, and in at least some cases, such as Ziconotide, intrathecal administration is required. We show herein that by coupling of a member of the family of α-conotoxins disclosed in PCT/AU02/00411 to AOD9604, oral bioavailability is achieved.

ACV peptides are neuronal nicotinic acetylcholine receptor antagonists. The effects of ACV peptides on noradrenaline and adrenaline release which is stimulated by nicotine were assessed as described in International Patent Application No. PCT/AU02/00411, with minor modifications. Adrenal chromaffin cells were isolated from adult bovine adrenal glands as described by Livett et al., (1987) In Poisner A M, Trifaro J M (eds) The Secretory Process, Vol 3. In-vitro methods for studying secretion. Elsevier, Amsterdam, 171-175 and 177-204. Isolated cells were plated out on collagen-coated 24-well plates at a density of 2.8×105 cells/cm².

Catecholamines were measured by electrochemical detection (650 mV BAS model LC-3A) following reversed-phase high-performance liquid chromatography.

Three- to four-day old cultured chromaffin cells were allowed to equilibrate to room temperature for 10 mins. The incubation medium was removed by two consecutive washes in HEPES buffer [150 mM NaCl, 2.6 mM KCl, 1.18 mM MgCl2.6H2O, 10 mM D-glucose, 10 mM Hepes free acid, 2.2 mM CaCl2.2H2O, 0.5% (w/v) bovine serum albumin, pH 7.4] for 10 min. Cells were then incubated with 1 μM, 5 μM or 10 μM of ACV1 or ACV3 for 10 min, before stimulation with 1-4 μM nicotine for a further 10 min. The incubation mixture was separated from the cells and acidified with 2M perchloric acid (PCA) to give a final concentration of 0.4M PCA. The catecholamines remaining in the chromaffin cells were released by lysing the cells with 0.4M PCA. Precipitated proteins were removed by centrifugation at 2500 rpm for 10 min. Basal release of catecholamines was measured in the presence of ACV peptide but the absence of nicotine. To determine the maximal release of catecholamines, a second control was stimulated with 1-4 μM nicotine in the absence of ACV peptide.

Catecholamines present in each sample were separated by reverse phase high performance liquid chromatography (RP-HPLC) utilizing a C18 column (Bio-Rad; 150 mm×4.6 mm, 5 μm particle size) and isocratic elution with 10% methanol in the mobile phase (70 mM KH2PO4, 0.1 mM NaEDTA, 0.2% heptane sulphonic acid). Catecholamines eluting from the column were identified by their retention time, and quantified by electrochemical detection (650 mV BAS model LC-3A). Known adrenaline and noradrenaline standards were used to calculate the amount of catecholamines in each sample, and these were expressed as a percentage of the total cell content.

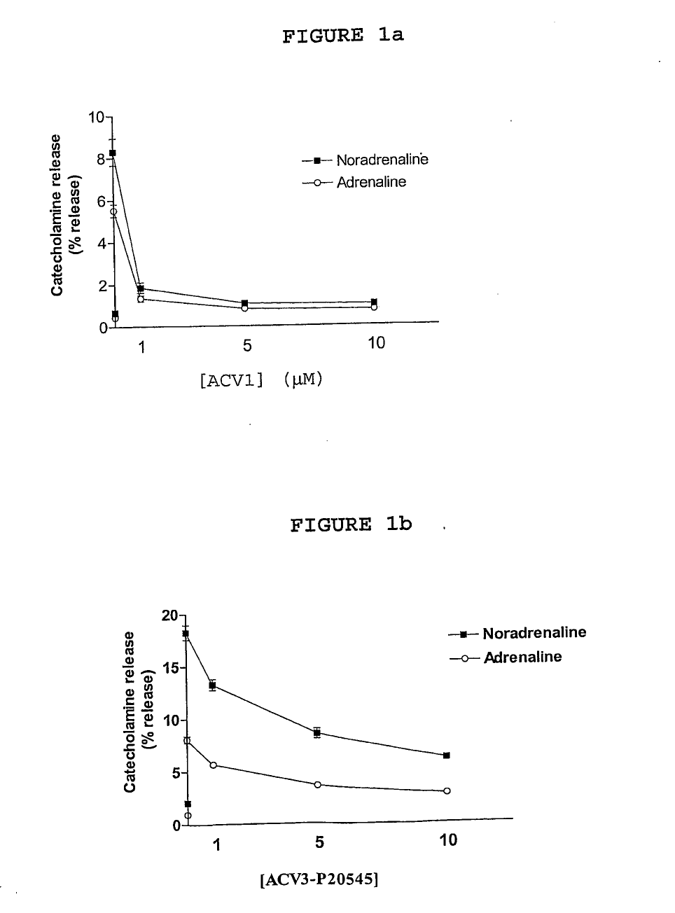

Peptides ACV1 and ACV3 were tested in this assay, and the results are summarized in FIG. 1. It can be seen that both peptides decreased catecholamine release in response to nicotine, confirming that the activity of the parent conotoxin ACV1 in this assay was retained at least in part by ACV3.

### Example 3

### ACV1 is not Effective when Administered Orally

The analgesic effect of ACV1 administered orally or sublingually was compared with the effect of subcutaneous administration in a model of chronic neuropathy in Sprague-Dawley rats. Chronic neuropathy was induced using a modified version of the chronic constriction injury (CCI) model of Bennett and Xie (1988) Pain 33, 87-107.

Under anaesthesia and using aseptic conditions, the right sciatic nerve in the mid-thigh region of the rat was exposed by blunt dissection through the biceps femoris muscle and separated from the surrounding connective tissues. For CCI groups, 4 ligatures (4-0 chromic gut) were loosely tied around the sciatic nerve so that they touched, but barely constricted the nerve. In all rats, the contralateral sides were not disturbed. The behaviour of the animals was observed after surgery to confirm recovery from anaesthesia.

The analgesic effect of ACV1 on mechanical pain threshold was assessed by measurement of mechanical paw withdrawal thresholds in conscious rats using a slightly modified version of the Randall-Selitto method (Randall and Selitto (1957) Arch. Int. Pharmacodyn. Ther. 111 409-419), with an Ugo Basile Analgesymeter (Varese, Italy). This instrument exerts a force which increases at a constant rate. A pointer moving along a linear scale continuously monitors this force. The force is applied to the rat's hind paw, which is placed on a small plinth under a cone-shaped pusher with a rounded tip (1.5 mm in diameter). The rat was held upright with the head and limb to be tested free, but with most of the rest of the body cradled in the hands of the experimenter. The paw was then put under the pusher until the rat withdrew the hind paw, or until 300 grams on the linear scale had elapsed.

A single dose of ACV1 was administered orally, sublingually or by subcutaneous injection to groups of rats as summarized in Table 3, and then hyperalgesia was tested 1 hour and 3 hours after administration. The oral administration was in a vehicle of saline and a gavage volume of 1 ml

TABLE 3
Treatment groups

| Treatment | | | Number of |
|---|---|---|---|
| group | Treatment | Route | animals |
| 1 | Placebo | Sub-cutaneous | 4 |
| 2 | ACV1 20 μg/kg | Sub-cutaneous | 6 |
| 3 | ACV1 100 μg/kg | Oral | 6 |
| 4 | ACV1 100 μg/kg | Sublingual | 6 |

N = number of rats in each group.

### Administration Volumes:

### Subcutaneous: 2.5 ml

### Oral: 1 ml

Sublingual: 35 μl (17.5 μl on either side of the tongue)

For oral administration, the rat was held with its head tilted backwards so that the neck is extended, and the distance from the nose to the xiphoid was measured to give an estimate of the length of the rounded end of the gavage tubing to be used. The needle gauge was determined by the viscosity of the substance to be administered.

For sublingual administration, the animal was anaesthetized prior to dosing, for example with a mixture of Ketamine (Pfizer Australia Pty Ltd)/Domitor or CO₂. The test agent was applied under each side of the tongue in volumes as stated above. Anaesthesia is reversed with Antisedan at 8 minutes, or after administration of the test agent.

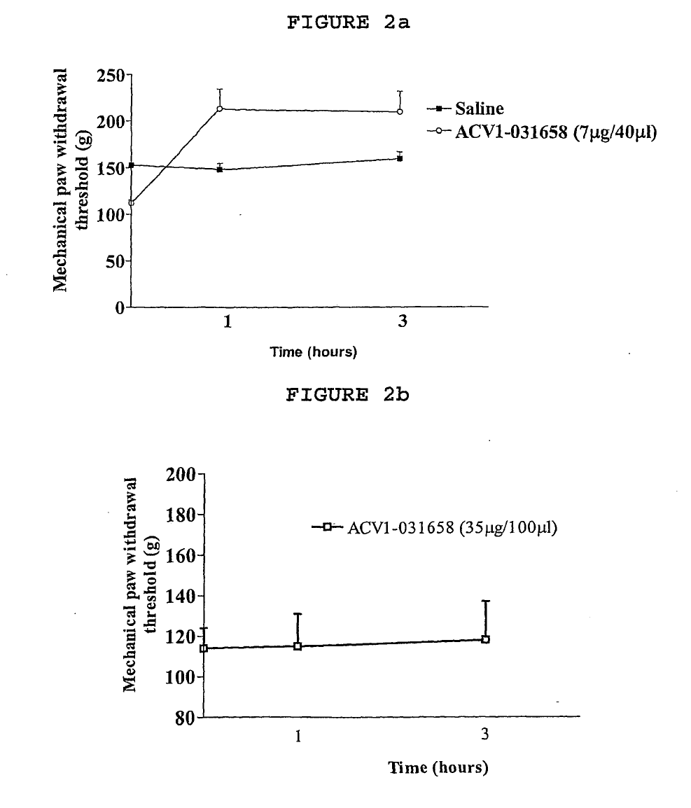

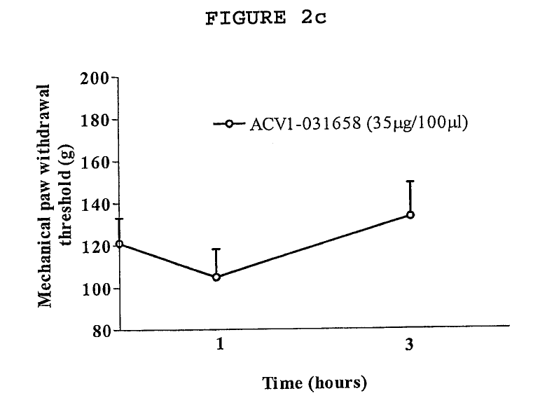

The results are summarized in FIG. 2. ACV1 was effective in relieving pain when administered subcutaneously (FIG. 2a). However, it was not effective when administered orally (FIG. 2c) or sublingually (FIG. 2b). ACV1 is therefore unlikely to be orally or sublingually available.

The expected low oral availability of ACV1 by the oral route was also confirmed by measuring plasma concentrations in dosed animals using an LC-MS/MS assay, for which the result was 1-2% compared with subcutaneous.

### Example 4

### ACV3 is Effective when Administered Sublingually

ACV3 was administered sublingually to rats with CCI and the analgesic effect it could potentially elicit was measured, as described in Example 3.

ACV3 at a dose of 0.96 mg/100 μl (2 mg/kg) was applied under both sides of the tongue of each animal. The vehicle was saline. This volume equates to 0.48 mg/54/1 each side. The method used to apply the drug sublingually does not necessitate anaesthetising the rats. All rats used in this experiment were conscious. The conscious state of the rats would probably have resulted in some, perhaps a large portion, of the drug making its way into the gut for possible absorption, as opposed to remaining in the mouth.

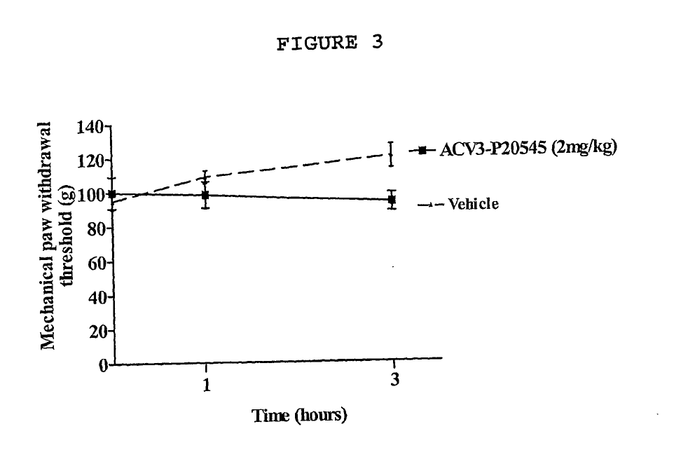

As shown in FIG. 3, there was a significant difference between sublingual ACV3 and the vehicle group, meaning that ACV3 had an analgesic effect when given sublingually. After one hour all animals treated with ACV3 showed an increase in withdrawal threshold, and after 3 hours this increase continued, with all but one animal showing a higher threshold at the 3 hour mark than at the one hour mark.

### Example 5

### ACV3 is Orally Effective in a Diabetic Neuropathic Pain Model

Outbred male Sprague-Dawley rats weighing 130-170 g (6-7 weeks old) were injected with streptozotocin (STZ; 75 mg/kg body weight dissolved in 0.1M sodium citrate buffer pH4) after an overnight fast. They were given 5% sucrose solution for 48 h and then placed on standard food (Barrostock GR2) and water ad libitum. A urine glucose assay (for example Diastix™, Bayer Australia Ltd) was conducted 2-3 days after diet change as an indication of the rat's diabetic status. Rats were then assessed for the level of hyperglycemia via a blood glucose assay (for example Reflolux S™, Boehringer Mannheim Australia Pty Ltd); blood was taken from the tail artery or vein. Rats with blood glucose values ≧27 mmol/l were included in this study. Six weeks after the STZ injection, animals were administered ACV3 (0.1 mg/kg, 0.3 mg/kg and 1 mg/kg) via either oral gavage or subcutaneous injection into the back of the neck and the analgesic effect it could potentially elicit was measured, as described in Example 3.

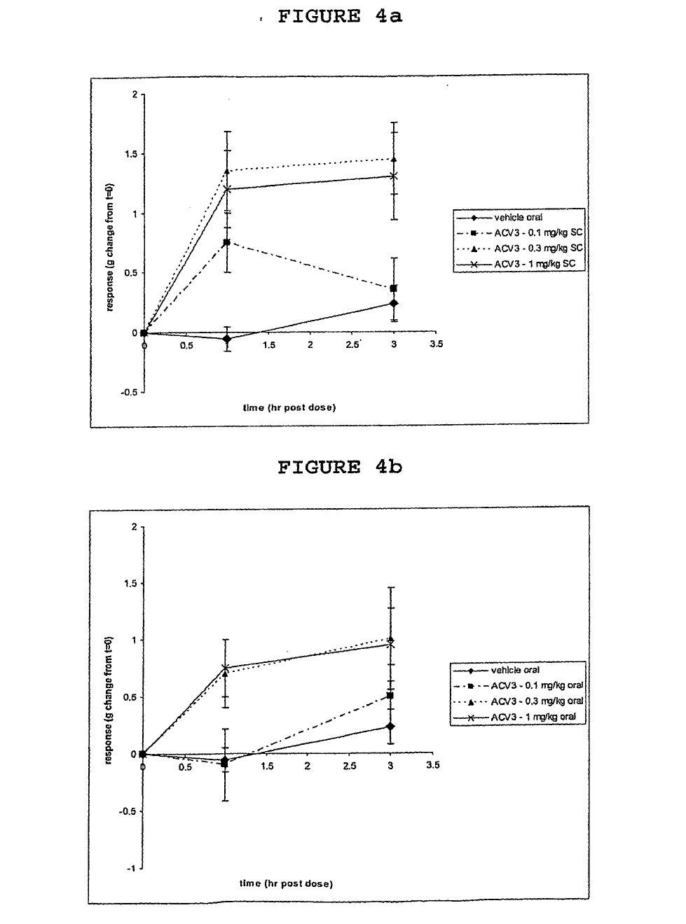

As seen in FIGS. 4a and 4b, both routes result in approximately the same minimum dose for maximal activity, 0.3 mg/kg. Based on the estimations of ED50 values from the dose response curves of ACV3 given orally and subcutaneously, ACV3 has a relative oral availability of 30-70%. Based on the time profile of effect, the oral absorption of ACV3 is rapid, and appears to reach the target tissues in a comparable time to subcutaneous delivery. The slightly higher maximal activity of ACV3 given subcutaneously compared with orally may relate to a different pattern of metabolites by the two routes.

### Example 6

### ACV3.2 is Orally Effective in a Diabetic Neuropathic Pain Model

The non-acetylated version of ACV3 (termed ACV3.2) was administered orally to diabetic rats (induced with streptozotocin as described above) with neuropathic pain and the anti-allodynic effect it could potentially elicit was measured, as described in Example 5.

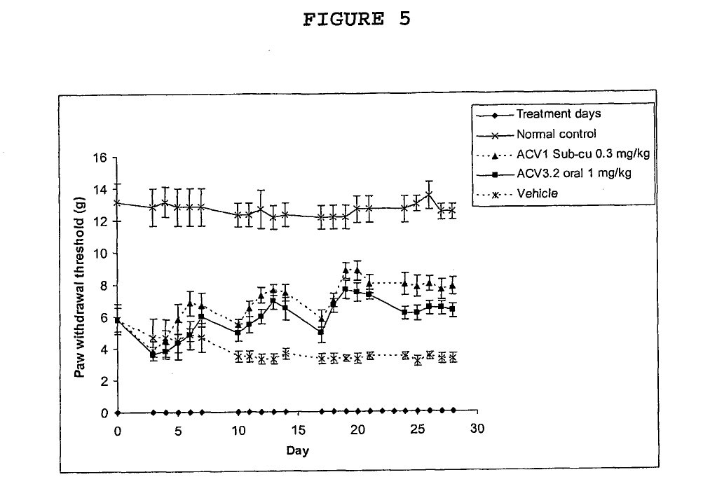

ACV3.2 at a dose of 1 mg/kg was administered daily via oral administration as described in Example 3, for 4 weeks. As shown in FIG. 5, there was a significant difference between ACV3.2 and the vehicle group, meaning hat ACV3.2 had an analgesic effect when given orally. Indeed the effect of ACV3.2 administered orally was similar to that of ACV1 administered via a subcutaneous injection. Therefore ACV3.2 also has substantial oral availability, probably similar to ACV3.

### Example 7

### EP-PTH is Effective at Increasing Blood Calcium Levels when Administered Orally

EP-PTH (SEQ ID NO: 14) is an analogue of parathyroid hormone, consisting of a fragment of the N-terminus of AOD9604 attached by its C-terminus to the N-terminus of parathyroid hormone.

Parathyroid hormone is known to regulate levels of blood calcium. However the low oral bioavailability of parathyroid hormone, reported to be about 0.3% precludes its use in osteoporosis. Here we show that, based on pharmacodynamic measurements EP-PTH has an oral bioavailability of approximately 10%.

EP-PTH was administered orally to normal mice and the anabolic action on blood calcium levels were measured. More specifically, after an initial blood sampling at time zero, groups of normal CD-1 mice were administered EP-PTH via either oral gavage (40 μg/kg, 200 μg/kg or 400 μg/kg) or subcutaneous injection (40 μg/kg) as described in Example 3. One hour after the administration of the EP-PTH and a second blood sample was taken. The serum calcium levels of blood samples were then assessed by an enzymatic method.

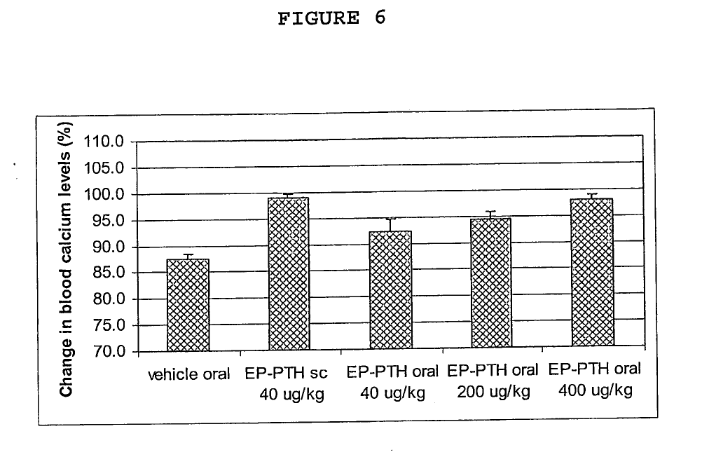

Orally administered EP-PTH showed a dose-dependent effect on blood calcium levels compared to the vehicle control. As shown in FIG. 6, animals administered 400 μg/kg of EP-PTH orally showed a similar effect on blood calcium levels to animals administered 40 μg/kg of EP-PTH via subcutaneous injection. This indicates that EP-PTH has a bioavailability of approximately 10%.

The person skilled in the art will understand that the method of estimating oral availability by identifying an oral dose response curve in a pharmacodynamic animal experiment and comparing with the parenterally administered dose response curve may be used in assessing the application of the invention to other parent peptides.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

It will be clearly understood that, although a number of prior art publications are referred to herein, and are incorporated by reference, this reference does not constitute an admission that any of these documents forms part of the common general knowledge in the art, in Australia or in any other country.

1. A method of improving the oral delivery of a parent peptide, comprising the step of linking the parent peptide to an added peptide to form a conjugate which has greater oral bioavailability than the parent molecule alone, the added peptide comprising a peptide of formula I
A-B-C (I)
in which A and C are each a hydrophobic amino acid residue or a substantially hydrophobic peptide of between 2 and 9 amino acid residues, A and C may be different and one of A or C may be absent
and B is one or more hydrophilic amino acid residues.

2. A peptide conjugate comprising a parent peptide linked to an added peptide, the added peptide comprising a peptide of formula I
A-B-C (I)
in which A and C are each a hydrophobic amino acid residue or a substantially hydrophobic peptide of between 2 and 9 amino acid residues, A and C may be different and one of A or C may be absent
and B is one or more hydrophilic amino acid residues.

3. A peptide conjugate according to claim 2 in which the conjugate has a maximum of 12 amino acids.

4. A peptide conjugate according to claim 2 in which A and or C is a hydrophobic amino acid residue.

5. A peptide conjugate according to claim 2 in which A and or C is a substantially hydrophobic peptide of 2 or 3 amino acid residue.

6. A peptide conjugate according to claim 2 in which B is 1 to 5 hydrophilic amino acid residues.

7. A peptide conjugate according to claim 2 in which B is 1 or 2 hydrophilic amino acid residues.

8. A peptide conjugate according to claim 6 or claim 7 in which one or more or all of the hydrophilic amino acid residues is a charged amino acid residue.

9. A peptide conjugate according to claim 2 in the added peptide has up to 12 amino acids.

10. A peptide conjugate according to claim 9 in the added peptide has up to 6 amino acids.

11. A peptide conjugate according to claim 10 in the added peptide is selected from SEQ Nos: 1 to 9.

12. A peptide conjugate according to claim 2 in which the parent peptide is a disulphide bonded peptide.

13. A peptide conjugate according to claim 2 in which the parent peptide has 20 or fewer amino acids.

14. A peptide conjugate according to claim 2 in which the parent peptide has 21 to 40 amino acids.

15. A peptide conjugate according to claim 2 in which the parent peptide has 41 to 60 amino acids.

16. A peptide conjugate according to claim 2 in which the parent peptide has 61 to 80 amino acids.

17. A peptide conjugate according to claim 2 in which the parent peptide has 81 or more amino acids.

18. A peptide conjugate comprising or consisting essentially of a peptide provided as one of SEQ ID NO: 12, 13 or 14.

19. A method according to claim 1 in which the peptide conjugate is according to any one of claims 2 to 18.

20. A pharmaceutical composition for oral administration, comprising a conjugate according to any one of claims 2 to 18, together with a pharmaceutically-acceptable carrier.

21. A method of treating a pathological disorder in an animal in need of treatment with a parent peptide, by orally administering to the animal an effective amount of a conjugate according to any one of claims 2 to 18 or a pharmaceutical composition according to claim 20, in which the parent peptide is not normally substantially orally bioavailable.

22. An oral delivery system comprising an added peptide, the added peptide comprising a peptide of formula I
A-B-C (I)
in which A and C are each a hydrophobic amino acid residue or a substantially hydrophobic peptide of between 2 and 9 amino acid residues, A and C may be different and one of A or C may be absent and B is one or more hydrophilic amino acid residues, wherein the added peptide is for conjugating to a parent peptide to improve the oral bioavailability of the parent peptide.

23. Use of a peptide comprising a peptide of formula I
A-B-C (I)
in which A and C are each a hydrophobic amino acid residue or a substantially hydrophobic peptide of between 2 and 9 amino acid residues, A and C may be different and one of A or C may be absent
and B is one or more hydrophilic amino acid residues, for linking to a parent peptide to improve the oral bioavailability of the parent peptide.

24. Use of an added peptide comprising a peptide of formula I
A-B-C (I)
in which A and C are each a hydrophobic amino acid residue or a substantially hydrophobic peptide of between 2 and 9 amino acid residues, A and C may be different and one of A or C may be absent
and B is one or more hydrophilic amino acid residues,
in the manufacture of a medicament comprising a parent peptide, in which the medicament is for administering orally to a patient in need of treatment with the parent peptide.
